(19) United States
(12) Patent Application Publication
(10) Pub. No.: US 2018/0168997 A1
(43) Pub. Date: Jun. 21, 2018

(54) HAIR CONDITIONING COMPOSITION

(71) Applicant: KAO GERMANY GMBH, DARMSTADT (DE)

(72) Inventors: Martin HOFFMANN, Darmstadt Hessen (DE); Jing NING, Darmstadt Hessen (DE)

(73) Assignee: KAO GERMANY GMBH, DARMSTADT (DE)

(21) Appl. No.: 15/737,049

(22) PCT Filed: Jun. 24, 2015
(86) PCT No.: PCT/EP2015/064290

§ 371 Date: Dec. 15, 2017

(51) Int. Cl. A61K 8/898; A61K 8/84; A61Q 5/12; A61K 8/39

(52) U.S. Cl. CPC A61K 8/898; A61K 8/84; A61K 2800/5422; A61K 8/39; A61K 2800/5426; A61Q 5/12

(57) ABSTRACT

The present invention relates to an aqueous composition for treating hair comprising one or more cationic polymers having certain cationic change density and one or more aminated silicones. The novel composition provides hair long lasting conditioning, especially improves combability and suppleness of hair which is held up to 20 hair washes.

The present invention relates to an aqueous composition for treating hair comprising one or more cationic polymers having certain cationic charge density and one or more aminated silicones. The novel composition provides hair long lasting conditioning, especially improves combability and suppleness of hair which is held up to 20 hair washes.

Hair conditioners are widely used for improving hair combability and suppleness among other properties which are as well improved and/or given. Consumers use the conditioners after each shampooing as hair combability and suppleness is usually reduced after cleansing the hair surface. Such process takes long time under shower and, therefore, there is a great need for hair conditioners which provides hair improved long lasting combability and suppleness. In other words, the consumers do not need to use a conditioning composition after each hair shampooing for improving combability and suppleness for certain number of hair washes.

The above mentioned problems have been subject of numerous research projects. The one approach has been improving hair conditioning properties of cleansing compositions so that there is no need for using of an additional composition only for making hair better combable and supple. In this way there have been many product initiatives on the market in form of so called two-in-one shampoo compositions.

DE 10 2013225898 discloses compositions for long lasting hair conditioning comprising cationic polymer with a relatively high charge density. It has been observed that compositions disclosed therein do not really provide the long lasting conditioning effects because of several reasons which are made clear below.

The inventors of the present invention have surprisingly find out that an aqueous composition comprising cationic quaternary ammonium polymer having certain cationic charge density and one or more aminated silicones provides hair long lasting conditioning effect, especially in terms of combability and suppleness, which is held with repeated hair washes up to 20 wash cycles.

Accordingly, the first object of the present invention is an aqueous composition for human hair comprising one or more cationic quaternary ammonium polymers having a cationic charge density of 3.0 mEq/g or more and one or more aminated silicones selected from the compounds according to the general structure

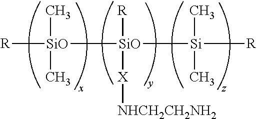

wherein R is the same or different OH or CH₃ or OCH₃ and X represents butyl, propyl, isopropyl or isobutyl.

Second object of the present invention is the use of the aqueous composition for long lasting conditioning of human hair, especially improving combability and suppleness of hair.

The third object of the present invention is a kit for treating human hair comprising the aqueous composition of the present invention and optionally a device which makes the increase of hair temperature possible.

The fourth object is the process for conditioning hair wherein the aqueous composition of the present invention is applied onto hair, optionally left on the hair for a period of 1 to 15 min, optionally rinsed off from hair and the hair temperature is increased to the range of 40 to 140° C.

The aqueous composition of the present invention comprises one or more cationic quaternary ammonium polymers having a charge density of 3 mEq/g or more, preferably 3.5 to 8 mEq/g, more preferably 4 to 7.5 mEq/g, most preferably 4.5 to 7.0 mEq and in particular 4.5 to 6.5 mEq/g. In a preferred embodiment of the present invention the aqueous composition comprises only one cationic quaternary ammonium polymer with the above defined cationic charge density ranges.

Suitable non-limiting cationic quaternary ammonium polymers for the purpose of the present invention are Polyquaternium-37, Polyquaternium-6, Polyquaternium-7 and Polyquaternium-16.

The preferred cationic polymers are Polyquaternium-37 and Polyquaternium-16. The particularly preferred cationic polymer is Polyquaternium-37

Total cationic quaternary ammonium polymer concentration having the above described cationic charge density is in the range of 0.1 to 5%, preferably 0.2 to 4%, more preferably 0.25 to 3% and most preferably 0.3 to 2.5% by weight, calculated to the total composition.

The aqueous composition of the present invention preferably comprises one or more aminated silicones. The suitable aminated silicones are the ones with primary or secondary or tertiary amine group bearing silicones. The quaternary ammonium group bearing silicones are not suitable as soon as their concentration exceeds 10%, preferably 5% of the total concentration of the one or more cationic polymer having charge density as defined above in the composition.

The especially preferred aminated silicone is Bis(Hydroxy/Methoxy) Amodimethicone wherein X is isobutyl and one of the R is OH and the other is OCH₃ in the above general structure.

Total concentration of one or more aminated silicones is in the range of 0.1 to 5%, preferably 0.2 to 4%, more preferably 0.25 to 3% and most preferably 0.3 to 2.5% by weight, calculated to the total composition.

The aqueous composition of the present invention preferably comprises one or more nonionic surfactants. Suitable ones are fatty alcohol ethoxylates of the following general structure

R₁(OCH₂CH₂)ₙOH

wherein R₁ is straight or branched, saturated or unsaturated alkyl chain which may be synthetic or natural with a C chain length in the range of 8 to 40, preferably 9 to 30 and more preferably 9 to 24 and n is a number in the range of 5 to 40, preferably 9 to 30.

Non-limiting suitable examples of the fatty alcohol ethoxylates are C9-11 Pareth-6, C9-11 Pareth-8, C9-15 Pareth-8, C11-13 Pareth-9, C11-13 Pareth-10, C11-15 Pareth-5, C11-15 Pareth-7, C11-15 Pareth-9, C11-15 Pareth-12, C11-15 Pareth-15, C11-15 Pareth-20, C11-15 Pareth-30, C11-15 Pareth-40, C11-21 Pareth-10, C12-13 Pareth-5, C12-13 Pareth-6, C12-13 Pareth-7, C12-13 Pareth-9, C12-13 Pareth-10, C12-13 Pareth-15, C12-13 Pareth-23, C12-14 Pareth-5, C12-14 Pareth-7, C12-14 Pareth-9, C12-14 Pareth-11, C12-14 Pareth-12, C12-15 Pareth-5, C12-15 Pareth-7, C12-15 Pareth-9, C12-15 Pareth-10, C12-15 Pareth-11, C12-15 Pareth-12, C12-16 Pareth-5, C12-16 Pareth-7, C12-16 Pareth-9, C13-15 Pareth-21, C14-15 Pareth-7, C14-15 Pareth-8, C14-15 Pareth-11, C14-15 Pareth-12, C14-15 Pareth-13, C20-22 Pareth-30, C20-40 Pareth-10, C20-40 Pareth-24, C20-40 Pareth-40, C20-40 Pareth-95, C22-24 Pareth-33, Beheneth-5, Beheneth-10, Beheneth-15, Beheneth-20, Beheneth-25, Beheneth-30, Ceteareth-5, Ceteareth-6, Ceteareth-7, Ceteareth-10, Ceteareth-11, Ceteareth-12, Ceteareth-15, Ceteareth-20, Ceteareth-25, Ceteareth-30, Ceteareth-35, Ceteareth-40, Laureth-5, Laureth-10, Laureth-15, Laureth-20, Laureth-25, Laureth-30, Laureth-40, Myreth-5, Myreth-10, Ceteth-5, Ceteth-10, Ceteth-15, Ceteth-20, Ceteth-25, Ceteth-30, Ceteth-40, Oleth-5, Oleth-10, Oleth-15, Oleth-20, Oleth-25, Oleth-30, Oleth-40, Steareth-5, Steareth-10, Steareth-15, Steareth-20, Steareth-25, Steareth-30, Steareth-35, and Steareth-40. They may also be comprised in the compositions as a mixture of more than one surfactant.

Further suitable nonionic surfactant is polypropylene glycol ether of fatty alcohols according to general structure

R₂(OCH₂(CH₃)CH₂)ₙOH

wherein R₂ is straight or branched, saturated or unsaturated fatty alcohol which may be synthetic or natural with a C chain length in the range of 8 to 40, preferably 9 to 30 and more preferably 9 to 24 and n is a number in the range of 1 to 40, preferably 3 to 30.

Suitable non-limiting examples are PPG-3 Caprylyl ether, PPG-5 Caprylyl ether, PPG-10 Caprylyl ether, PPG-10 Cetyl ether, PPG-20 Cetyl ether, PPG-28 Cetyl ether, PPG-30 Cetyl ether, PPG-7 Lauryl ether, PPG-10 Lauryl ether, PPG-10 Oleyl ether, PPG-20 Oleyl ether, PPG-23 Oleyl ether, PPG-30 Oleyl ether, PPG-11 Stearyl ether and PPG-15 Stearyl ether.

Further suitable nonionic surfactants are polyethylene glycol fatty acid esters of the following general structure

R₃C(O)(OCH₂CH₂)ₙOH

wherein R₃ is straight or branched, saturated or unsaturated alkyl group which may be synthetic or natural with a C chain length in the range of 7 to 39, preferably 9 to 29 and more preferably 9 to 23 and n is a number in the range of 5 to 40, preferably 9 to 30.

Suitable non-limiting examples are PEG-8 Behenate, PEG-8 Caprate, PEG-8 Caprylate, PEG-5 Cocoate, PEG-8 Cocoate, PEG-9 Cocoate, PEG-10 Cocoate, PEG-15 Cocoate, PEG-6 Isopalmitate, PEG-6 Isostearate, PEG-8 Isostearate, PEG-9 Isostearate, PEG-10 Isostearate, PEG-12 Isostearate, PEG-20 Isostearate, PEG-30 Isostearate, PEG-40 Isostearate, PEG-6 Laurate, PEG-8 Laurate, PEG-9 Laurate, PEG-10 Laurate, PEG-12 Laurate, PEG-14 Laurate, PEG-20 Laurate, PEG-30 Laurate, PEG-8 Myristate, PEG-20 Myristate, PEG-5 Oleate, PEG-6 Oleate, PEG-7 Oleate, PEG-8 Oleate, PEG-9 Oleate, PEG-10 Oleate, PEG-11 Oleate, PEG-12 Oleate, PEG-15 Oleate, PEG-20 Oleate, PEG-30 Oleate, PEG-32 Oleate, PEG-6 Palmitate, PEG-18 Palmitate, PEG-20 Palmitate, PEG-5 Stearate, PEG-6 Stearate, PEG-7 Stearate, PEG-8 Stearate, PEG-9 Stearate, PEG-10 Stearate, PEG-12 Stearate, PEG-14 Stearate, PEG-15 Stearate, PEG-20 Stearate, PEG-25 Stearate, PEG-30 Stearate, PEG-35 Stearate and PEG-40 Stearate.

Further suitable nonionic surfactants are polypropylene glycol fatty acid esters of the following general structure

R₄C(O)(OCH₂(CH₃)CH₂)ₙOH

wherein R₄ is straight or branched, saturated or unsaturated alkyl group which may be synthetic or natural with a C chain length in the range of 7 to 39, preferably 9 to 29 and more preferably 9 to 23 and n is a number in the range of 1 to 40, preferably 9 to 30.

Suitable non-limiting examples are PPG-15 Isostearate, PPG-9 Laurate, PPG-26 Oleate and PPG-36 Oleate.

Further nonionic suitable surfactants are polyethylene glycol and polypropylene glycol ether of fatty alcohols of the following general structure

R₅(OCH₂(CH₃)CH₂)ₙ₁(OCH₂CH₂)ₙ₂OH

wherein R₅ is straight or branched, saturated or unsaturated alkyl group which may be synthetic or natural with a C chain length in the range of 7 to 39, preferably 9 to 29 and more preferably 9 to 23 and n1 and n2 may be the same or different and are a number in the range of 1 to 40.

Suitable non-limiting examples are PPG-2 Ceteareth-9, PPG-4 Ceteareth-12, PPG-4 Ceteareth-20, PPG-2 C9-11 Pareth-5, PPG-2 C9-11 Pareth-7, PPG-2 C9-11 Pareth-8, PPG-2 C9-11 Pareth-11, PPG-2 C12-13 Pareth-8, PPG-2 C12-15 Pareth-6, PPG-4 C 13-15 Pareth-15, PPG-5 C9-15 Pareth-6, PPG-6 C9-11 Pareth-5, PPG-6 C12-15 Pareth-12, PPG-6 C12-18 Pareth-11, PPG-1 Deceth-4, PPG-1 Deceth-5, PPG-1 Deceth-6, PPG-1 Deceth-7, PPG-2 Deceth-3, PPG-2 Deceth-7, PPG-2 Deceth-8, PPG-2 Deceth-10, PPG-2 Deceth-15, PPG-2 Deceth-20, PPG-2 Deceth-30, PPG-2 Deceth-40, PPG-4 Deceth-4, PPG-4 Deceth-6, PPG-4 Deceth-6, PPG-6 Deceth-4, PPG-6 Deceth-9, PPG-8 Deceth-6, PPG-14 Deceth-6, PPG-2 Laureth-5, PPG-2 Laureth-8, PPG-2 Laureth-12, PPG-3 Laureth-8, PPG-3 Laureth-9, PPG-3 Laureth-10, PPG-3 Laureth-12, PPG-4 Laureth-2, PPG-4 Laureth-5, PPG-4 Laureth-7, PPG-4 Laureth-15, PPG-5 Laureth-5, PPG-5 Laureth-3, PPG-20 Laureth-12, PPG-25 Laureth-25, PPG-3 Myreth-3, PPG-3 Myreth-11, PPG-9 Steareth-3, PPG-23 Steareth-34, PPG-30 Steareth-4, PPG-34 Steareth-3, and PPG-38 Steareth-6.

Further suitable nonionic surfactants are ethoxylated triglycerides. Well known and commonly used ones are ethoxylated castor oil such as PEG-40 hydrogenated castor oil or and PEG-60 hydrogenated castor oil.

Total concentration of non-ionic surfactants is in the range of 0.1 to 5%, preferably 0.2 to 4%, more preferably 0.25 to 3% and most preferably 0.3 to 2.5% by weight, calculated to the total composition.

In a preferred embodiment of the present invention the aqueous composition of the present invention does not comprise quaternary ammonium surfactants at high concentrations. It has been observed that when quaternary ammonium surfactants are comprised in the composition at a concentration above 10% preferably 5% of the total concentration of the one or more cationic polymers as defined above, the long lasting effect of the aqueous composition is dramatically diminished. Without being bound by the theory, one explanation might be that the amount of adsorbed/bound cationic polymer concentration is decreased by co-adsorption of the cationic surfactants. Accordingly, the aqueous composition of the present invention does not comprise quaternary ammonium surfactants at a concentration of 10%, preferably 5% or more, by weight, of the total concentration of cationic polymer as defined above.

Specifically, the aqueous composition of the present invention does not comprise quaternary ammonium surfactant of the general structure below at a concentration more than 10%, preferably 5% by weight of the total concentration of one or more cationic quaternary ammonium polymers having the cationic charge density as defined above. The general structure of cationic quaternary ammonium surfactant is

R₁₁R₁₂R₁₃R₁₄N

wherein R₁₁ is an alkyl chain having C length of 8 to 30 which may be saturated or unsaturated, straight or branched, Rue an alkyl chain having C length of 1 to 30 which may be saturated or unsaturated, straight or branched, R₁₁ and R₁₂ additionally may take the structures of

R₁₅C(O)O(CH2)n or R₁₅C(O)NH(CH2)n

wherein R15 is an alkyl chain with a C length of 7 to 29 which may be saturated or unsaturated, straight or branched and n is a number between 1 and 4, R₁₃ and R₁₄ is an alkyl chain with a C length of 1 to 4 which may be straight or branched (only for C3 and C4), wherein all alkyl chains may include one or more substituents such as hydroxyl or (poly)ethoxy groups.

The composition of the present invention may comprise one or more fatty alcohols and may be in a form of emulsion. Suitable non-limiting examples are according to general structure

R₆—OH

wherein R₆ is straight or branched, saturated or unsaturated alkyl chain which may be synthetic or natural with a C chain length in the range of 12 to 24 which may as well be substituted with one or more groups on the alkyl chain.

Non limiting suitable examples are lauryl alcohol, myristyl alcohol, cetyl alcohol, oleyl alcohol, stearyl alcohol, behenyl alcohol and their mixtures. As a mixed fatty alcohol the mostly used one is the cetearyl alcohol as well the preferred fatty alcohol in the compositions of the present invention.

The total concentration of one or more fatty alcohols is in the range of 0.1 to 20%, preferably 0.5 to 15%, more preferably 1 to 10% and the most preferably 2 to 7.5% by weight calculated to the total composition.

The aqueous composition of the present invention may comprise one or more organic solvents. Suitable ones are ethanol, propanol, isopropanol, benzyl alcohol, benzyloxyethanol, ethoxydiglycol, alkylene carbonates such as ethylene carbonate and propylene carbonate, phenoxyethanol, butanol, isobutanol, cyclohexane, cyclohexanol, hexyleneglycol, ethylenecarbonate, propyleneglycol, poypropyleneglycols, ethyleneglycol monoethylether, ethylene glycol monobutyl ether, ethylene glycol monophenyl ether, 1-phenylethylalcohol, 2-phenylethylalcohol, o-methoxyphenol. The most preferred ones are ethanol, isopropanol, benzylalcohol and polypropylene glycols. Concentration of organic solvents should not exceed 20% by weight, preferably in the range of 0.1 to 15%, more preferably 0.1 to 10% by weight and most preferably 0.1 to 7.5% by weight calculated to total composition. It should be noted that concentration of one or more organic solvents is very much dependent on the type of preparation i.e. a solution can contain higher concentration of organic solvent than a gel or emulsion composition.

Further in an embodiment of the present invention, the aqueous composition of the present invention comprises at least one hair direct dye selected from cationic and neutral nitro dyes.

Any cationic direct dye is in principal suitable for the compositions. Non-limiting suitable examples are Basic Blue 6, Basic Blue 7, Basic Blue 9, Basic Blue 26, Basic Blue 41, Basic Blue 99, Basic Blue 124, Basic Brown 4, Basic Brown 7, Basic Brown 16, Basic Brown 17, Basic Green 1, Basic Orange 31, Basic Red 2, Basic Red 12, Basic Red 22, Basic Red 51, Basic Red 76, Basic Violet 2, Basic Violet 3, Basic Violet 10, Basic Violet 14, Basic Violet 57 and Basic Yellow 87.

Neutral dyes, so called nitro dyes for shading purposes are also optionally contained in the compositions. Suitable ones are HC Blue No. 2, HC Blue No. 4, HC Blue No. 5, HC Blue No. 6, HC Blue No. 7, HC Blue No. 8, HC Blue No. 9, HC Blue No. 10, HC Blue No. 11, HO Blue No. 12, HO Blue No. 13, HO Brown No. 1, HO Brown No. 2, HO Green No. 1, HO Orange No. 1, HO Orange No. 2, HO Orange No. 3, HO Orange No. 5, HO Red BN, HO Red No. 1, HO Red No. 3, HO Red No. 7, HO Red No. 8, HO Red No. 9, HO Red No. 10, HO Red No. 11, HO Red No. 13, HO Red No. 54, HO Red No. 14, HO Violet BS, HO Violet No. 1, HO Violet No. 2, HO Yellow No. 2, HO Yellow No. 4, HO Yellow No. 5, HO Yellow No. 6, HO Yellow No. 7, HO Yellow No. 8, HO Yellow No. 9, HO Yellow No. 10, HO Yellow No. 11, HO Yellow No. 12, HO Yellow No. 13, HO Yellow No. 14, HO Yellow No. 15, 2-Amino-6-chloro-4-nitrophenol, picramic acid, 1,2-Diamino-4-nitrobenzol, 1,4-Diamino-2-nitrobenzol, 3-Nitro-4-aminophenol, 1-Hydroxy-2-amino-3-nitrobenzol and 2-hydroxyethylpicramic acid.

Total concentration of one or more direct dyes in total is in the range of 0.001 to 1.0% by weight, preferably 0.01 to 0.8% more preferably 0.05 to 0.75% and most preferably 0.1 to 0.5% by weight calculated to total composition.

The aqueous composition of the present invention may further comprise one or more UV filters which may be selected from water soluble ones as well as oils soluble ones. The oil soluble UV filter are more preferred ones as they show no interaction with the cationic quaternary ammonium polymers. Non-limiting examples are 4-Aminobenzoic acid and the esters and salts thereof, 2-phenyl benzimidazole-5-sulfonic acid and the alkali and amine salts thereof, 4-dimethyl aminobenzoic acid and the esters and salts thereof, cinnamic acid and the esters and salts thereof, 4-methoxycinnamic acid and the esters and salts thereof, salicylic acid and the esters and salts thereof, 2.4-dihydroxybenzophenone, 2.2′.4.4′-tetrahydroxy-benzophenone, 2-hydroxy-4-methoxybenzophenone and its 5-sulfonic acid or the sodium salt thereof, 2.2′-dihydroxy-4.4′-dimethoxybenzophenone, 2-hydroxy-5-chlorobenzophenone, 2.2′-dihydroxy-4-methoxybenzophenone, 2.2′-dihydroxy-4.4′-dimethoxy-5.5′-disulfobenzo-phenone or the sodium salt thereof, 2-hydroxy-4-octyloxybenzophenone, 2-hydroxy-4-methoxy-4′-methylbenzophenone, 3-benzyl-idenecampher, 3-(4′-sulfo)-benzyl-idenebornane-2-one and the salts thereof, 3-(4′-methyl benzylidene)-DL-campher, and/or polysilicone-15.

The total UV filter concentration may be in the range of 0.01 to 1% by weight calculated to the total composition.

The composition used in the process of the present invention may comprise oil and/or oily substances which are suitably selected from silicone oils, either volatile or non-volatile, natural and synthetic oils, fatty alcohol ethers (dialkyl ethers) and fatty acid fatty alcohol esters. Among silicone oils those can be added to the compositions include, dimethicone, dimethiconol, polydimethylsiloxane (DC fluid ranges from Dow Corning), arylated silicones such as phenyl methicone, phenyl trimethicone, diphenyl dimethicone, diphenylsiloxy phenyl trimethicone, tetramethyl tetraphenyl trisiloxane, triphenyl trimethicone, and trimethyl pentaphenyl trisiloxane, aqueous emulsion of divinyldimethicone/dimethicone copolymer, preferably with a viscosity of higher than 1×10⁸ mm²/s, more preferably higher than 1.1×10⁸ mm²/s measured at 0.01 Hz and at approximately 25° C.

Suitable non-limiting examples to natural plant oils are such as olive oil, almond oil, avocado oil, wheatgerm oil and ricinus oil.

Suitable non-limiting examples to fatty alcohol fatty acid esters are such as isopropyl myristate, palmitate, stearate and isostearate, oleyl oleate, isocetyl stearate, hexyl laurate, dibutyl adipate, dioctyl adipate, myristyl myristate and oleyl erucate.

Suitable non-limiting examples to fatty alcohol ethers (dialkyl ethers) are such as dimyristyl ether, dicetyl ether and dicaprylyl ether.

The compositions comprise oil and/or oily substances at a total concentration in the range of 0.01 to 10%, preferably 0.05 to 7.5%, more preferably 0.1 to 5 and most preferably 0.1 to 3% by weight calculated to total composition.

The compositions according to the invention may also comprise further conditioning substances such as protein hydrolyzates and polypeptides, e.g., keratin hydrolyzates, collagen hydrolyzates of the type “Nutrilan^{R}” or elastin hydrolyzates, as well as also in particular plant protein hydrolyzates, optionally, cationized protein hydrolyzates, e.g., “Gluadin^{R}”.

The composition of the present invention may further comprise natural plant extracts. Within the meaning of the present invention the extracts are liquid extracts and prepared by mixing plant parts such as leaves, fruits, blossoms and roots with a solvent such as water, alcohol, propyleneglycol or mixture of more than one solvent and incubating for certain period of time and filtrating the undissolved plant parts. Suitable aqueous (e.g. steam-distilled) alcoholic or hydro-alcoholic plant extracts known per se are in particular aloe, pineapple, artichoke, arnica, avocado, valerian, bamboo, henbane, birch, stinging nettle, echinacea, ivy, wild angelica, gentian, ferns, pine needles, silver weed, ginseng, broom, oat, rose hip, hamamelis, hay flowers, elderberry, hop, coltsfoot, currants, chamomile, carrots, chestnuts, clover, burr root, cocoanut, cornflower, lime blossom, lily of the valley, marine algae, balm, mistletoe, passion flower, ratanhia, marigold, rosemary, horse chestnut, pink hawthorn, sage, horsetail, yarrow, primrose, nettle, thyme, walnut, wine leaves, white hawthorn, etc. Suitable trade products are, for example, the various “Extrapon®” products, “Herbasol^{R}”, “Sedaplant^{R}” and “Hexaplant^{R}”. Extracts and the preparation thereof are also described in “Hagers Handbuch der pharmazeutischen Praxis”, 4^{th} Ed. Preferred plant extracts are prepared from Vitis vinifera, Malus domestica, Camelia sinensis, Juglans regia Ribes Uva-Crispa, Ribes nigrum, Ribes rubrum and Punica granatum. The above mentioned extracts may also be available in the powder form and such are also suitable within the meaning of the present invention.

The natural plant extracts are included into the compositions at a concentration of 0.001 to 1.0%, preferably 0.005 to 0.75%, more preferably 0.01 to 0.6% and most preferably 0.05 to 0.5% by weight, calculated to total composition based on dry matter of the extract.

Further in an embodiment of the present invention, compositions comprise one or more ubiquinone of the following general structure

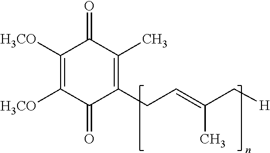

where n is a number between 1 and 10. It should be noted that the compositions of the present invention can certainly comprise more than one ubiquinone. Preferred ubiquinones are the ones where n is a number between 6 and 10 and especially preferred is Ubiquinone 50 where n is 10, also known as Coenzyme Q10.

Concentration ubiquinone of the above formula in the compositions is from 0.0001 to 1%, preferably from 0.0002 to 0.75%, more preferably from 0.0002 to 0.5% and most preferably from 0.0005 to 0.5% by weight, calculated to total composition.

The pH of the compositions according to the present invention is suitably between 3.0 and 8.0 and preferably in the range of 3.5 to 6.5, more preferably 4.0 to 5.5 and most preferably 4.0 to 5.

In principal pH of the compositions can be adjusted with any organic and/or inorganic acids or their mixture. Some of them to mention are phosphoric acid, hydrochloric acid as the inorganic ones and to the organic acids the well-known citric acid and lactic acid, glycolic acid, glyoxylic acid, hydroxyacrylic acid, glyceric acid, malic acid and tartaric acid and of the dicarboxylic acids are malonic acid, succinic acid, glutaric acid, adipic acid, maleic acid, fumaric acid and phtalic acid.

Conditioning compositions of the present invention can comprise additionally any compound customarily found in conditioning compositions such as chelating agents, preservatives and fragrance.

The sequestering agents are selected from polycarboxy acids. The preferred one is ethylene diamine tetraacetic acid, EDTA. Typical useful concentration range for sequestering agents is of 0.01-2.5% by weight calculated to the total composition.

Viscosity of the conditioning composition may be adjusted according to the application form and generally should not be more than 50,000 mPa·s at 20° C. measured with Brookfield Rheometer at a shear rate of 5 sec⁻¹.

Thickening agents especially the nonionic thickening polymers may be comprised into the compositions of the present invention. Suitable non-limiting examples are cellulose derivatives such as hydroxyethyl cellulose, methyl cellulose, hydroxymethyl cellulose, hydroxypropyl cellulose, guar gum and its derivatives, and cognac mannan and derivatives. Thickeners may be included at a concentration of 0.05 to 2.5% by weight calculated to total composition. Concentration of thickener is very much dependent on the thickener itself and also the preparation such as pH value of the composition etc. and therefore should be selected depending on the desired viscosity of the composition.

The aqueous composition of the present invention may be in the form of solution, gel, dispersion and emulsion. These preparations may be provided to the consumers as they are confectioned in a suitable packaging which allows easy release of the products and distribution onto hair as well as they may be provided in a pressurized container. In case pressurized container is used as the vessel carrying the composition, it additionally comprises a pressurizing gas which may be selected form the any known ones such as alkanes, butane, isobutene, dimethyl ether, pressurized air, nitrogen and carbon dioxide.

The compositions of the present invention is used in a process for long lasting conditioning hair wherein the hair temperature is increased to the range of 40 to 140° C., preferably 60 to 140° C., more preferably 70 to 130° C., most preferably 80 to 120° C. and in particular 90 to 110° C. The heating of the hair is achieved with a hair dryer, with digital hair heating devices, with an iron which may be either flat or having certain surface structures or a round shaped iron. The operation of such kind of devices is commonly known by the skilled in the art or by the users who may be seen as average users of such devices.

In a preferred embodiment of the process the aqueous composition is not rinsed off from the hair prior to increasing the temperature of the hair.

The following examples are to illustrate the invention but not to limit it.

### EXAMPLE 1

The following compositions were prepared for the comparative tests.

| | % active matter | | |
|---|---|---|---|
| | A | B | C |
| Polyquaternium 37 | 0.8 | 0.8 | 0.8 |
| C12-13 Pareth-9 | — | 0.5 | 0.5 |
| PPG-3 Caprylyl ether | — | — | 1.0 |
| Bis(Hydroxy/Methoxy) | — | — | 0.5 |
| Amodimethicone | | | |

| | |
|---|---|
| Citric acid | q.s. to pH 4.0 |
| Water | to100 |

The composition C is within the scope of the claim 1.

Human hair streaks having approximately 25 cm length and weighing approximately 3.5 g was used for the comparative tests. The streaks were damaged prior to the tests by means of double bleaching with a bleaching composition commercially available under the brand name Goldwell.

The streaks, always in duplicate, were treated with the above compositions. Therefore, each streak was applied approximately 1 g of the product and left on the streak for 15 min and the streaks were rinsed off with water. Afterwards only one of the streaks of each pair was dried with a hair drier. Hair temperature reached was approximately 95° C. The temperature measurement was carried out using an infrared measuring device from a distance of approximately 5 cm.

Additionally two hair streaks were treated with composition C and the one was heated using hair drier to a temperature of 95° C. without rinsing off the composition (C+* in the Table below) and the other was treated with an hot iron operated at 130° C. for 5 times which resulted in approximate hair temperature of 100° C. (C+** in the Table below).

The suppleness of the hair was measured with a zwick machine and expressed as the force (in milliNewton-mN) to pull off a streak through the rods of the machine. The machine is commercially available from Zwick/Roell Company and includes a manual. It was used according to its manual. Briefly, the streak was placed among 5 metal rods which are placed offset in a given distance horizontally (2 cm) and vertically (8 cm) 5 in total and the force needed to pull the streaks through the metal rods at a given rate (5 cm/min) was measured. All values reported in the Table below are average of at least 6 readings.

In order to test the long lastingness of hair suppleness, the hair streaks were washed 5 times first and the suppleness was measured again as described above and after additional 5 washes the measurements were repeated. For washing hair streaks, 5% by weight Sodium lauryl ether sulfate solution in water at pH 5.0 was used.

The following results were obtained.

Suppleness (mN)

| Composition | Heat | Start | 5 Wash | 10 Wash |
|---|---|---|---|---|
| Water | − | 1741 | 2072 | 1938 |
| A | + | 756 | 873 | 1023 |
| A | − | 791 | 1145 | 1578 |
| B | + | 697 | 809 | 798 |
| B | − | 715 | 1046 | 1311 |
| C | + | 450 | 515 | 659 |
| C | − | 525 | 1020 | 1280 |
| C | +* | 348 | 518 | 629 |
| C | +** | 388 | 489 | 572 |

It should be noted that the lower the value, the higher the suppleness of the hair.

From the above results it is clear that the aqueous composition according to the present invention improves hair suppleness and this effect is long lasting especially when hair temperature is increased after application of the composition. It is beyond any doubt that the addition of aminated silicone to Polyquaternium-37 comprising composition improved the suppleness of the hair. It was observed that the suppleness was affected by the presence of additional substances but the streaks treated with the composition of the present invention were judged to have enough suppleness.

The above results prove beyond any doubt the improved suppleness of the hair treated with the aqueous composition of the present invention.

The following examples fall within the scope of the present invention.

### EXAMPLE 2

| | % by weight |
|---|---|
| Polyquaternium-37 | 0.80 |
| C12-13 Pareth-9 | 0.50 |
| Bis(Hydroxy/Methoxy) Amodimethicone | 0.50 |
| Hydroxyethylcellulose | 0.50 |
| Lactic acid | q.s. to pH 4.2 |
| Water | to 100 |

### EXAMPLE 3

| | % by weight |
|---|---|
| Polyquaternium-37 | 0.80 |
| C12-13 Pareth-9 | 0.50 |
| Bis(Hydroxy/Methoxy) Amodimethicone | 0.50 |
| Hydroxypropyl guar | 0.50 |
| Lactic acid | q.s. to pH 4.2 |
| Water | to 100 |

### EXAMPLE 4

| | % by weight |
|---|---|
| Polyquaternium-37 | 0.80 |
| C12-13 Pareth-9 | 0.50 |
| Bis(Hydroxy/Methoxy) Amodimethicone | 0.50 |
| Basic Red 51 | 0.20 |
| Basic Orange 31 | 0.02 |
| Basic Yellow 87 | 0.08 |
| Citric acid | q.s. to pH 3.8 |
| Water | to 100 |

### EXAMPLE 5

| | % by weight |
|---|---|
| Polyquaternium-37 | 0.80 |
| C12-13 Pareth-9 | 0.85 |
| Bis(Hydroxy/Methoxy) Amodimethicone | 0.50 |
| Benzophenone-3 | 0.25 |
| Basic Red 51 | 0.20 |
| Basic Orange 31 | 0.02 |
| Basic Yellow 87 | 0.08 |
| Citric acid | q.s. to pH 3.8 |
| Water | to 100 |

### EXAMPLE 6

| | % by weight |
|---|---|
| Polyquaternium-37 | 0.80 |
| C12-13 Pareth-9 | 0.85 |
| Bis(Hydroxy/Methoxy) Amodimethicone | 0.50 |
| Benzophenone-3 | 0.25 |
| Ethylhexyl methoxy cinnamon | 0.10 |
| Basic Red 51 | 0.20 |
| Basic Orange 31 | 0.02 |
| HC red no 3 | 0.08 |
| Citric acid | q.s. to pH 3.8 |
| Water | to 100 |

### EXAMPLE 7

| | % by weight |
|---|---|
| Polyquaternium-37 | 0.80 |
| C12-13 Pareth-9 | 0.85 |
| Bis(Hydroxy/Methoxy) Amodimethicone | 0.50 |
| Benzophenone-3 | 0.25 |
| Ethylhexyl methoxy cinnamate | 0.10 |
| Hydroxypropyl guar gum | 0.25 |
| Basic Red 51 | 0.20 |
| Basic Orange 31 | 0.02 |
| HC red no 3 | 0.08 |
| Citric acid | q.s. to pH 3.8 |
| Water | to 100 |

### EXAMPLE 8

| | % by weight |
|---|---|
| Polyquaternium-37 | 0.80 |
| Ceteareth 20 | 1.25 |
| Cetearyl alcohol | 5.00 |
| Bis(Hydroxy/Methoxy) Amodimethicone | 0.50 |
| Dimethicone 100 cSt | 0.85 |
| Ethylhexyl methoxy cinnamate | 0.10 |
| Lactic acid | q.s. to pH 3.8 |
| Water | to 100 |

### EXAMPLE 9

| | % by weight |
|---|---|
| Polyquaternium-37 | 0.80 |
| Ceteareth 30 | 1.25 |
| Cetearyl alcohol | 2.00 |
| Behenyl alcohol | 2.80 |
| Dimethicone 50 cSt | 0.85 |
| Almond oil | 0.10 |
| Vitis vinifera extract | 0.10 (dry matter) |
| Lactic acid | q.s. to pH 4.5 |
| Water | to 100 |

### EXAMPLE 10

| | % by weight |
|---|---|
| Polyquaternium-37 | 0.80 |
| Ceteareth 30 | 1.25 |
| Cetearyl alcohol | 2.00 |
| Behenyl alcohol | 2.80 |
| Basic Red 51 | 0.20 |
| Basic Orange 37 | 0.02 |
| HC red no 3 | 0.08 |
| Basic yellow 87 | 0.03 |
| HC Yellow 2 | 0.03 |
| Bis(Hydroxy/Methoxy) Amodimethicone | 0.85 |
| Almond oil | 0.10 |
| Vitis vinifera extract | 0.10 (dry matter) |
| Lactic acid | q.s. to pH 4.5 |
| Water | to 100 |

1. An aqueous composition for human hair comprises one or more cationic quaternary ammonium polymers having a cationic charge density of 3.0 mEq/g or more and one or more aminated silicone selected from compounds according to general structure 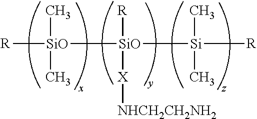
wherein R is the same or different OH or CH₃ or OCH₃ and X represents butyl, propyl, isopropyl or isobutyl.

2. The composition according to claim 1 wherein the cationic quaternary ammonium polymer has a charge density of 3.5 to 8 mEq/g.

3. The composition according to claim 1 wherein the cationic quaternary ammonium polymer is selected from Polyquaternium-37, Polyquaternium-6, Polyquaternium-7, and Polyquaternium-16.

4. The composition according to claim 1 wherein it comprises one or more cationic quaternary ammonium polymers at a total concentration of 0.1 to 5% by weight calculated to the total composition.

5. The composition according to claim 1 wherein the aminated silicone is Bis(Hydroxy/Methoxy) Amodimethcone.

6. The composition according to claim 1 wherein it comprises one or more aminated silicones at a total concentration in the range of 0.1 to 5% by weight calculated to the total composition.

7. The composition according to claim 1 wherein one or more nonionic surfactants are selected from
a—fatty alcohol ethoxylates of the following general structure
R1(OCH2CH2)nOH
wherein R1 is straight or branched, saturated or unsaturated alkyl chain which may be synthetic or natural with a C chain length in the range of 8 to 40 and n is a number in the range of 5 to 40.
b—polypropylene glycol ether of fatty alcohols according to general structure
R2(OCH2(CH3)CH2)nOH
wherein R2 is straight or branched, saturated or unsaturated fatty alcohol which may be synthetic or natural with a C chain length in the range of 8 to 40 and n is a number in the range of 1 to 40
c—polyethylene glycol fatty acid esters of the following general structure
R3C(O)(OCH2CH2)nOH
wherein R3 is straight or branched, saturated or unsaturated alkyl group which may be synthetic or natural with a C chain length in the range of 7 to 39 and n is a number in the range of 5 to 40
d—polypropylene glycol fatty acid esters of the following general structure
R4C(O)(OCH2(CH3)CH2)nOH
wherein R4 is straight or branched, saturated or unsaturated alkyl group which may be synthetic or natural with a C chain length in the range of 7 to 39 and n is a number in the range of 1 to 40
e—polyethylene glycol and polypropylene glycol ether of fatty alcohols of the following general structure
R5(OCH2(CH3)CH2)n1(OCH2CH2)n2OH
wherein R5 is straight or branched, saturated or unsaturated alkyl group which may be synthetic or natural with a C chain length in the range of 7 to 39 and n1 and n2 may be the same or different and are a number in the range of 1 to 40.
f—ethoxylated triglycerides.

8. The composition according to claim 1 wherein it comprises one or more nonionic surfactant at a total concentration of 0.1 to 5% by weight, calculated to the total composition.

9. The composition according to claim 1 wherein it does not comprise quaternary ammonium surfactant at a concentration more than 10% by weight of the total concentration of one or more cationic quaternary ammonium polymers.

10. The composition according to claim 1 wherein it comprises one or more of the following compounds
Organic solvent,
Fatty alcohol of the general structure
R6-OH
wherein R6 is straight or branched, saturated or unsaturated alkyl chain which may be synthetic or natural with a C chain length in the range of 12 to 24 which may as well be substituted with one or more groups on the alkyl chain,
UV filter,
Hair direct dye selected from cationic and neutral nitro dyes,
Natural plant extract,
Protein hydrolyzate,
Ubiquinone, and
Nonionic polymer.
Oil and/or oily substances selected from silicone oils, natural and synthetic oily, fatty alcohol dialkyl ethers and fatty acid fatty alcohol esters.

11. The composition according to claim 1 wherein it has a pH in the range of 3.0 to 8.0.

12. (canceled)

13. (canceled)

14. The process according to claim 13 wherein the hair is heated up with a hair dryer, with a digital hair heating device, with an iron which may be either flat or having certain surface structures or a round shaped iron.

15. (canceled)
